(19) United States
(12) Patent Application Publication
(10) Pub. No.: US 2008/0287402 A1
(43) Pub. Date: Nov. 20, 2008

(54) Use of a glial attenuator to prevent amplified pain responses caused by glial priming

(76) Inventors: Kirk W. Johnson, Alameda, CA (US); Linda R. Watkins, Denver, CO (US)

Correspondence Address: ROBINS & PASTERNAK LLP, 1731 EMBARCADERO ROAD, SUITE 230, PALO ALTO, CA, 94303, US

(21) Appl. No.: 12/150,944

(22) Filed: May 1, 2008

Related U.S. Application Data
(60) Provisional application No. 60/927,334, filed on May 3, 2007.

(51) Int. Cl. A61K 31/437; A61P 17/00; A61K 31/65

(52) U.S. Cl. 514/154; 514/300

(57) ABSTRACT

The use of a glial attenuator, such as ibudilast (3-isobutyryl-2-isopropylpyrazolo[1,5-a]pyridine), to prevent the negative consequences of glial priming is described. In particular, the present invention is directed to a method of treating a subject with ibudilast to prevent amplified pain responses to inflammation or injury as a result of glial priming following an initial glial activating event.

### CROSS-REFERENCE TO RELATED APPLICATIONS

This application claims the benefit under 35 U.S.C. §119(e)(1) to U.S. Provisional Application Ser. No. 60/927,334, filed May 3, 2007, which application is incorporated herein by reference in its entirety.

### STATEMENT REGARDING FEDERALLY SPONSORED RESEARCH OR DEVELOPMENT

This invention was made with support under NIDA Grant R01 DA017670, “Pain facilitation via neuron-to-glia signaling” and NIDA Grant K02 DA015642, “Immune/glial mediation of exaggerated pain states.” Accordingly, the United States Government may have certain rights in this invention.

### FIELD OF THE INVENTION

The present invention relates generally to the use of a glial attenuator, such as ibudilast (3-isobutyryl-2-isopropylpyrazolo[1,5-a]pyridine; also termed AV411 herein), to prevent the negative consequences of glial priming. In particular, the present invention is directed to a method of treating a subject with ibudilast to prevent amplified pain responses to inflammation or injury as a result of glial priming following an initial glial activating event.

### BACKGROUND OF THE INVENTION

In recent years, pain management has become an area of increasing focus in the medical profession, partly due to the growing population of elderly, issues surrounding quality of life, and the growing numbers of patients reportedly suffering from pain. Pain is both a sensory and emotional experience, and is generally associated with tissue damage or inflammation. Typically, pain is divided into two general categories—acute pain and chronic pain. Both differ in their etiology, pathophysiology, diagnosis, and most importantly, treatment.

Acute pain is short term, and is typically of a readily identifiable cause. Patients suffering from acute pain typically respond well to medications. In contrast, chronic pain, medically-defined as pain that lasts for 6 months or longer, is often not associated with an obvious injury; indeed, patients can suffer from protracted pain that persists for months or years after the initial insult. While acute pain is generally favorably treated with medications, chronic pain is often much more difficult to treat, generally requiring expert care. Reportedly, according to the American Chronic Pain Association, over 86 million Americans suffer from chronic pain, and the management of chronic pain has long been recognized as an unmet clinical need.

Glial priming may contribute to the transition between “normal pain” and chronic pain in an individual. After becoming activated in response to a pain-evoking event, glial cells, including microglia and astrocytes, can return either to a normal basal state that has little influence on pain responsivity or a “primed” state that is over-responsive to new challenges (Watkins et al. (2007) Brain, Behavior, and Immunity 21:131-146). Primed glia respond more rapidly and release more glial-derived neuroexcitatory substances (e.g., proinflammatory cytokines, chemokines, ATP, excitatory amino acids, and nitric oxide) than glia in the basal state. Glia can become primed in response to a variety of challenges, such as infection, inflammation, ischemia, neurodegeneration, or injury to tissue. After an initial glial priming event (e.g., inflammation or injury), a later pain-evoking event leads to remarkably stronger and longer enhanced pain responses than if the prior glial activation event had not occurred. Thus, glial priming contributes to the severity and duration of later pain episodes.

Most chronic pain is neuropathic in nature (also referred to as neuralgia), i.e., pain that occurs as a result of injury or disease to the nerve tissue itself, and accounts for large numbers of patients presenting to an ever-growing number of pain clinics with chronic, non-malignant pain. Neuropathic pain (NP) is a maladaptive chronic condition in which pain originates from damaged nerves, often yielding pain that is out-of-proportion to the extent of injury. NP is thought to result, at least in part, from glial activation in the central nervous system of mammals (Watkins and Maier (2004) Drug Disc. Today: Ther. Strategies 1(1): 83-88). While affecting wide demographics, it is most prevalent in patients of advanced age, and therefore its occurrence is rising in the United States. The pain suffered by these patients is variously described as burning, stabbing, and shock-like, and can persist long after the initiating insult has healed. In addition to these sensory conditions, many patients experience heightened sensitivity to innocuous stimuli (allodynia). Moreover, such pain is notoriously difficult to treat.

Neuropathic pain is an important component of a number of syndromes of varying etiologies whose common characteristic is the development of a prolonged and profound pain state. Among these conditions are the viral neuralgias (e.g., herpes, AIDS), diabetic neuropathy, phantom limb pain, stump/neuroma pain, post-ischemic pain (stroke), fibromyalgia, reflex sympathetic dystrophy (RSD), complex regional pain syndrome (CRPS), cancer pain, vertebral disk rupture, trigeminal neuralgia, and others.

Unfortunately, neuropathic pain management in this patient population is at best inconsistent, if not oftentimes ineffective, due in part to the fact that pain is subjective, and clinical training in the area of pain management is generally inadequate. Current first-line treatments for chronic pain include opioids, analgesics such as gabapentin, and tricyclic antidepressants. In the instance of opioids, when administered over prolonged periods, undesirable side effects such as drug tolerance, chemical dependency and even physiological addiction can occur. Of treatment regimes currently available for chronic pain, at best, approximately 30% are effective in significantly diminishing the pain, and may lose their efficacy over time. Although numerous pharmacological agents are available for the treatment of neuropathic pain, a definitive therapy has remained elusive.

In instances in which treatment with a single agent proves to be unsuccessful, combination therapy is often then explored as a second line treatment. For example, such combination therapy may employ administration of an opioid agent with an adjuvant analgesic, although the relative doses of each are often subject to prolonged trial and error periods. Oftentimes, triple drug therapy is necessary. Such therapy generally involves a combination of tricyclic antidepressants, anti-convulsants, and a systemic local anesthetic. Patient compliance drops significantly, however, when treatment requires the administration of multiple pharmacologic agents. Recently, researchers reported the use of a combination of morphine and gabapentin in a randomized study for controlling nerve pain (Gilron, I., et al., New Eng J. of Medicine, Vol 352:1281-82, No. 13, Mar. 31, 2005).

The small molecule, ibudilast, (3-isobutyryl-2-isopropylpyrazolo[1,5-a]pyridine), is a non-selective inhibitor of cyclic nucleotide phosphodiesterase (PDE) (Fujimoto, T., et al., J. of Neuroimmunology, 95 (1999) 35-92). Ibudilast also acts as an LTD4 antagonist, an anti-inflammatory, a PAF antagonist, and a vasodilatatory agent (Thompson Current Drug Reports). Ibudilast is thought to exert a neuroprotective role in the central nervous system of mammals, presumably via suppression of the activation of glial cells (Mizuno et al. (2004) Neuropharmacology 46: 404-411). Ibudilast has been widely used in Japan for relieving symptoms associated with ischemic stroke or bronchial asthma. Marketed indications for ibudilast in Japan include its use as a vasodilator, for treating allergy, eye tissue regeneration, ocular disease, and treatment of allergic ophthalmic disease (Thompson Current Drug Reports). In recent clinical trials, its use in the treatment of multiple sclerosis, an inflammatory disease of the central nervous system, has been explored (News. Medical. Net; Pharmaceutical News, 2 Aug. 2005). While the use of ibudilast for a number of varying indications has been reported to date, to the best of the applicants' knowledge, its use in treating amplified pain associated with glial priming has heretofore remained largely unexplored.

In light of the shortcomings in current approaches for treating pain, there exists a need for improved compositions and methods for treating pain, particularly to counteract enhanced pain responses associated with glial priming following an initial glial-activating event.

### SUMMARY OF THE INVENTION

In one aspect, the invention provides a method for inhibiting glial priming in a subject resulting from a glial-activating event, comprising administering to the subject an effective amount of ibudilast. In one embodiment, the subject is human. In certain embodiments, the glial-activating event is tissue injury, infection, inflammation, aging and/or chronic opioid use in the subject. In one embodiment, the ibudilast is administered at about the same time as or before the glial-activating event. In another embodiment, the ibudilast is administered after the glial-activating event. In a further embodiment, the ibudilast is administered before and after the glial-activating event. In yet another embodiment, the ibudilast is administered at about the same time as the glial-activating event and after the glial-activating event. In preferred embodiments, the ibudilast is administered about 1-5 days before and about 1-5 days after the glial-activating event. In one embodiment, the ibudilast is administered two days before and five days after the glial-activating event.

In certain embodiments, ibudilast is administered systemically, for example, via intravenous, subcutaneous, intraperitoneal, oral, intranasal, sublingual, intramuscular or other systemic routes. In other embodiments, ibudilast is administered centrally, for example, intrathecally. In other embodiments, the ibudilast is administered topically. In certain embodiments, multiple therapeutically effective doses of the ibudilast are administered to the subject.

In another aspect, the invention provides a method for preventing or diminishing amplified pain resulting from glial priming after a first glial-activating event, the method comprising administering to a subject in need thereof a therapeutically effective amount of ibudilast, wherein amplified pain after a second glial-activating event is diminished or eliminated. In certain embodiments, the first glial-activating event is tissue injury, infection or inflammation. In one embodiment, the ibudilast is administered at about the same time as or before the first glial-activating event. In another embodiment, the ibudilast is administered after the first glial-activating event. In a further embodiment, the ibudilast is administered before and after the first glial-activating event. In yet another embodiment, the ibudilast is administered at about the same time as the first glial-activating event and after the first glial-activating event. In preferred embodiments, the ibudilast is administered about 1-5 days before and about 1-5 days after the first glial-activating event. In one embodiment, the ibudilast is administered two days before and five days after the first glial-activating event. In certain embodiments, the method further comprises administering ibudilast at about the same time as or after the second glial activating event.

Mammalian subjects suitable for treatment by the methods described herein include, but are not limited to, those suffering from inflammation, chronic pain, neuropathic pain, post-operative pain, injury-related pain, or disease-related pain. In one embodiment, the subject is a human.

In certain embodiments, ibudilast is administered systemically, for example, via intravenous, subcutaneous, intraperitoneal, oral, intranasal, sublingual, intramuscularly or other systemic routes. In other embodiments, ibudilast is administered centrally, for example, intrathecally. In other embodiments, the ibudilast is administered topically.

A therapeutic dosage amount of ibudilast may be achieved by intermittent administration, or administration once daily (i.e., in a single dose), twice daily (i.e., in two separate doses), three times daily, or may be administered as multiple doses over a time course of several days, weeks, or even months. Such administering is typically over a duration of time effective to result in a diminution, and ideally elimination or even reversal, of pain, including amplified pain resulting from glial priming. Exemplary durations of treatment include at least about one week, from 1 week to 1 month, from two weeks to 2 months, up to about 6 months, up to about 12 months or even longer. In one particular embodiment, treatment lasts from about 1 week to about 50 weeks. In a preferred embodiment of the treatment method, the administering is over a duration of time effective to result in elimination of pain.

In certain embodiments, the method further comprises administering one or more other glial attenuators. Exemplary glial attenuators that may also be used in the practice of the invention include, but are not limited to, Minocycline, Fluorocitrate, MW01-5-188WH, Propentofylline (also a PDE inhibitor), Pentoxyfylline (also a PDE inhibitor), Rolipram (also a PDE inhibitor), IL-10, IL-1 receptor antagonist(s), TNF-receptor antagonist(s) including sTNFR, MAP-kinase inhibitor(s), Yohimbine, glial cell chloride antagonists, caspase inhibitors, MMP inhibitors, cannabinoid receptor (e.g., type 2) agonists, arundic acid, statins, thalidomide and related analogs.

In certain embodiments, the method further comprises administering one or more other agents effective for treating pain or opioid withdrawal syndrome. Such agents include analgesics, non-steroidal anti-inflammatory drugs (NSAIDs), antiemetics, antidiarrheals, alpha-2-antagonists, benzodiazepines, anticonvulsants, antidepressants, and insomnia therapeutics. In various embodiments, one or more agents are selected from the group consisting of buprenorphine, naloxone, methadone, levomethadyl acetate, L-alpha acetylmethadol (LAAM), hydroxyzine, diphenoxylate, atropine, chlordiazepoxide, carbamazepine, mianserin, benzodiazepine, phenoziazine, disulfuram, acamprosate, topiramate, ondansetron, sertraline, bupropion, amantadine, amiloride, isradipine, tiagabine, baclofen, propranolol, tricyclic antidepressants, desipramine, carbamazepine, valproate, lamotrigine, doxepin, fluoxetine, imipramine, moclobemide, nortriptyline, paroxetine, sertraline, tryptophan, venlafaxine, trazodone, quetiapine, zolpidem, zopiclone, zaleplon, gabapentin, memantine, pregabalin, cannabinoids, tramadol, duloxetine, milnacipran, naltrexone, paracetamol, metoclopramide, loperamide, clonidine, lofexidine, and diazepam.

In another aspect, the invention provides a composition or combination effective for treating pain, including amplified pain resulting from glial priming. The composition comprises ibudilast and optionally one or more additional agents effective for treating pain or opioid withdrawal syndrome, wherein each of the components is either contained in a single composition or dosage form (such as in an admixture), or is present as a discrete or separate entity (e.g., in a kit). A composition of the invention may optionally include one or more pharmaceutically acceptable excipients.

In yet another aspect, the invention encompasses a kit comprising ibudilast, for the treatment of pain, including amplified pain resulting from glial priming, and optionally, one or more additional agents effective for treating pain or opioid withdrawal syndrome, for simultaneous, sequential or separate use.

These and other embodiments of the subject invention will readily occur to those of skill in the art in view of the disclosure herein.

### BRIEF DESCRIPTION OF THE FIGURES

FIG. 1 compares the responses to pain in the von Frey test for untreated rats and rats treated with ibudilast (AV411) to prevent amplified pain resulting from glial priming following a first injury (laparotomy). Pain was monitored after a second injury (urinary bladder inflammation from cyclophosphamide induced cystitis) occurring 2 weeks after the first injury.

### DETAILED DESCRIPTION OF THE INVENTION

The practice of the present invention will employ, unless otherwise indicated, conventional methods of chemistry, biochemistry, and pharmacology, within the skill of the art. Such techniques are explained fully in the literature. See, e.g.; A. L. Lehninger, Biochemistry (Worth Publishers, Inc., current addition); Morrison and Boyd, Organic Chemistry (Allyn and Bacon, Inc., current addition); J. March, Advanced Organic Chemistry (McGraw Hill, current addition); Remington: The Science and Practice of Pharmacy, A. Gennaro, Ed., 20^{th} Ed.; Goodman & Gilman The Pharmacological Basis of Therapeutics, J. Griffith Hardman, L. L. Limbird, A. Gilman, 10^{th} Ed.

All publications, patents and patent applications cited herein, whether supra or infra, are hereby incorporated by reference in their entirety.

### I. DEFINITIONS

In describing and claiming the present invention, the following terminology will be used in accordance with the definitions described below.

It must be noted that, as used in this specification and the intended claims, the singular forms “a,” “an,” and “the” include plural referents unless the context clearly dictates otherwise. Thus, for example, reference to “a drug” includes a single drug as well as two or more of the same or different drugs, reference to “an optional excipient” refers to a single optional excipient as well as two or more of the same or different optional excipients, and the like.

“Pharmaceutically acceptable excipient or carrier” refers to an excipient that may optionally be included in the compositions of the invention and that causes no significant adverse toxicological effects to the patient.

“Pharmaceutically acceptable salt” includes, but is not limited to, amino acid salts, salts prepared with inorganic acids, such as chloride, sulfate, phosphate, diphosphate, hydrobromide, and nitrate salts, or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly salts containing pharmaceutically acceptable cations include, but are not limited to, sodium, potassium, calcium, aluminum, lithium, and ammonium (including substituted ammonium).

“Active molecule” or “active agent” as described herein includes any agent, drug, compound, composition of matter or mixture which provides some pharmacologic, often beneficial, effect that can be demonstrated in-vivo or in vitro. This includes foods, food supplements, nutrients, nutriceuticals, drugs, vaccines, antibodies, vitamins, and other beneficial agents. As used herein, the terms further include any physiologically or pharmacologically active substance that produces a localized or systemic effect in a patient.

“Substantially” or “essentially” means nearly totally or completely, for instance, 95% or greater of some given quantity.

“Optional” or “optionally” means that the subsequently described circumstance may or may not occur, so that the description includes instances where the circumstance occurs and instances where it does not.

“Glial priming” refers to glial cells, which after becoming activated in response to a challenge (e.g., pain-evoking event), return to a “primed” state that is over-responsive to new challenges. Primed glia respond more rapidly and release more glial-derived neuroexcitatory substances (e.g., proinflammatory cytokines, chemokines, ATP, excitatory amino acids, and/or nitric oxide) than glia in a normal basal state. Glia can become primed in response to a “glial activating event” such as, but not limited to, infection, inflammation, ischemia, neurodegeneration, aging, chronic opioid use, or injury to tissue. Glial priming causes amplified pain, that is, increased severity and/or duration of a later pain episode.

“Amplified pain” or “enhanced pain” means after an initial glial activating event, a later pain-evoking event results in stronger and longer pain responses than if the prior glial activation event had not occurred.

By “pathological pain” is meant any pain resulting from a pathology, such as from functional disturbances and/or pathological changes, lesions, burns and the like. One form of pathological pain is “neuropathic pain” which is pain caused by nerve damage. Examples of pathological pain include, but are not limited to, thermal or mechanical hyperalgesia, thermal or mechanical allodynia, diabetic pain, pain arising from irritable bowel or other internal organ disorders, endometriosis pain, phantom limb pain, complex regional pain syndromes, fibromyalgia, low back pain, cancer pain, pain arising from infection, inflammation or trauma to peripheral nerves or the central nervous system, multiple sclerosis pain, entrapment pain, and the like.

“Hyperalgesia” means an abnormally increased pain sense, such as pain that results from an excessive sensitiveness or sensitivity.

“Hypalgesia” (or “hypoalgesia”) means the decreased pain sense.

“Allodynia” means pain that results from a non-noxious stimulus to the skin. Examples of allodynia include, but are not limited to, cold allodynia, tactile allodynia, and the like.

“Nociception” is defined herein as pain sense. “Nociceptor” herein refers to a structure that mediates nociception. The nociception may be the result of a physical stimulus, such as, mechanical, electrical, thermal, or a chemical stimulus. Nociceptors are present in virtually all tissues of the body.

“Analgesia” is defined herein as the relief of pain without the loss of consciousness.

An “analgesic” is an agent or drug useful for relieving pain, again, without the loss of consciousness.

The term “central nervous system” or “CNS” includes all cells and tissue of the brain and spinal cord of a vertebrate. Thus, the term includes, but is not limited to, neuronal cells, glial cells (astrocytes, microglia, oligodendrocytes), cerebrospinal fluid (CSF), interstitial spaces and the like.

“Glial cells” refer to various cells of the CNS also known as microglia, astrocytes, and oligodendrocytes.

The terms “subject”, “individual” or “patient” are used interchangeably herein and refer to a vertebrate, preferably a mammal. Mammals include, but are not limited to, murines, rodents, simians, humans, farm animals, sport animals and pets.

The term “about”, particularly in reference to a given quantity, is meant to encompass deviations of plus or minus five percent.

The terms “effective amount” or “pharmaceutically effective amount” of a composition or agent, as provided herein, refer to a nontoxic but sufficient amount of the composition to provide the desired response, such as suppression of glial priming in a subject, and optionally, a corresponding therapeutic effect, such as preventing, diminishing, or eliminating pain amplification in a subject. The exact amount required will vary from subject to subject, depending on the species, age, and general condition of the subject, the severity of the condition being treated, the particular drug or drugs employed, mode of administration, and the like. An appropriate “effective” amount in any individual case may be determined by one of ordinary skill in the art using routine experimentation.

“Treatment” or “treating” pain includes: (1) preventing pain, i.e. causing pain not to develop or to occur with less intensity in a subject that may be exposed to or predisposed to pain but does not yet experience or display pain, (2) inhibiting pain, i.e., arresting the development or reversing pain, or (3) relieving pain, i.e., decreasing the amount of pain experienced by the subject.

By “therapeutically effective dose or amount” of ibudilast is intended an amount that, when ibudilast is administered as described herein, brings about a positive therapeutic response in treatment of pain, such as preventing, diminishing, or eliminating pain in a subject, particularly amplified pain resulting from glia priming due to a prior injury, inflammation, or other glia activating event.

### II. MODES OF CARRYING OUT THE INVENTION

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

The present invention is based on the discovery that the glial attenuator, ibudilast, can be used to prevent amplified pain responses resulting from glial priming after an initial glial-activating event. As shown in Example 1, administration of ibudilast to subjects after a first injury (laparotomy) reduced the severity and duration of pain in subjects after a second injury (urinary bladder inflammation from cyclophosphamide induced cystitis).

Although not wishing to be bound by theory, glia can adopt basal, activated and primed states. Glia can become primed in response to a variety of challenges, such as infection, inflammation, ischemia, neurodegeneration, or injury to tissue. In the primed state, glia are over-reactive to new challenges. Primed glia respond more rapidly and release more glial-derived neuroexcitatory substances (e.g., proinflammatory cytokines, chemokines, ATP, excitatory amino acids, and/or nitric oxide) than glia in a normal basal state. Glial priming can lead to amplified pain, that is, increased severity and/or duration of a later pain episode. Thus, an apparently identical trauma can cause rapidly resolved pain in some patients if glia are initially in the basal state, but persistent or even chronic pain in others as a result of glial priming.

In order to further an understanding of the invention, a more detailed discussion is provided below regarding methods of treating pain with ibudilast, particularly to prevent amplification of pain as a result of glial priming.

Treatment with Ibudilast to Prevent Pain Amplification

The invention relates to the use of a glial attenuator, such as ibudilast, to inhibit glial priming that can lead to amplification of pain in a subject during a later pain-evoking event and the development of chronic pain. Ibudilast has been shown in the present application to reduce the severity and duration of pain in subjects after a second injury following a previous glial-activating event (see Example 1).

In certain embodiments, the invention provides a method for preventing or diminishing amplified pain resulting from glial priming after a first glial-activating event, the method comprising administering to a subject in need thereof a therapeutically effective amount of ibudilast, wherein amplified pain after a second glial-activating event is diminished or eliminated. In certain embodiments, the subject is administered an effective amount of ibudilast at about the same time as the first glial-activating event, preferably about 1-5 days before and/or about 1-5 days after the first glial-activating event. In one embodiment, the ibudilast is administered two days before and five days after the first glial-activating event. Ibudilast may also be administered shortly before, during, or after the second glial activating event. Mammalian subjects suitable for treatment by the methods described herein include, but are not limited to, those suffering from inflammation, chronic pain, neuropathic pain, post-operative pain, injury-related pain, or disease-related pain.

In certain embodiments, the method further comprises administering one or more other glial attenuators in addition to ibudilast. Exemplary glial attenuators that may also be used in the practice of the invention include, but are not limited to, Minocycline, Fluorocitrate, MW01-5-188WH, Propentofylline (also a PDE inhibitor), Pentoxyfylline (also a PDE inhibitor), Rolipram (also a PDE inhibitor), IL-10, IL-1 receptor antagonist(s), TNF-0801-0066 receptor antagonist(s) including sTNFR, MAP-kinase inhibitor(s), Yohimbine, glial cell chloride antagonists, caspase inhibitors, MMP inhibitors, cannabinoid receptor (e.g., type 2) agonists, arundic acid, statins, thalidomide and related analogs.

In certain embodiments, a therapeutically effective amount of ibudilast is administered to a subject in combination therapy with one or more other agents for treating pain or opioid withdrawal syndrome. Such agents include, but are not limited to, analgesics, NSAIDs, antiemetics, antidiarrheals, alpha-2-antagonists, benzodiazepines, anticonvulsants, antidepressants, and insomnia therapeutics. Exemplary agents include, but are not limited to, buprenorphine, naloxone, methadone, levomethadyl acetate, L-alpha acetylmethadol (LAAM), hydroxyzine, diphenoxylate, atropine, chlordiazepoxide, carbamazepine, mianserin, benzodiazepine, phenoziazine, disulfuram, acamprosate, topiramate, ondansetron, sertraline, bupropion, amantadine, amiloride, isradipine, tiagabine, baclofen, propranolol, tricyclic antidepressants, desipramine, carbamazepine, valproate, lamotrigine, doxepin, fluoxetine, imipramine, moclobemide, nortriptyline, paroxetine, sertraline, tryptophan, venlafaxine, trazodone, quetiapine, zolpidem, zopiclone, zaleplon, gabapentin, memantine, pregabalin, cannabinoids, tramadol, duloxetine, milnacipran, naltrexone, paracetamol, metoclopramide, loperamide, clonidine, lofexidine, and diazepam.

### Pharmaceutical Compositions

Ibudilast

Ibudilast is a small molecule drug (molecular weight of 230.3) having the structure shown below.

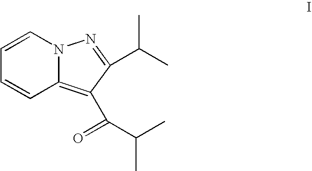

Ibudilast is also found under ChemBank ID 3227, CAS #50847-11-5, and Beilstein Handbook Reference No. 5-24-03-00396. Its molecular formula corresponds to [C₁₄H₁₈N₂O]. Ibudilast is also known by various chemical names which include 2-methyl-1-(2-(1-methylethyl)pyrazolo(1,5-a)pyridin-3-yl)1-propanone; 3-isobutyryl-2-isopropylpyrazolo(1,5-a)pyridine]; and 1-(2-isopropyl-pyrazolo[1,5-a]pyridin-3-yl)-2-methyl-propan-1-one. Other synonyms for ibudilast include Ibudilastum (Latin), BRN 0656579, KC-404, and the brand name Ketas®. Ibudilast, as referred to herein, is meant to include any and all pharmaceutically acceptable salt forms thereof, prodrug forms (e.g., the corresponding ketal), and the like, as appropriate for use in its intended formulation for administration.

Ibudilast is a non-selective nucleotide phosphodiesterase (PDE) inhibitor (most active against PDE-3, PDE-4, PDE-10, and PDE-11 (Gibson et al. (2006) Eur. J. Pharmacology 538:39-42)), and has also been reported to have LTD4 and PAF antagonistic activities. Its profile appears effectively anti-inflammatory and unique in comparison to other PDE inhibitors and anti-inflammatory agents. PDEs catalyze the hydrolysis of the phosphoester bond on the 3′-carbon to yield the corresponding 5′-nucleotide monophosphate. Thus, they regulate the cellular concentrations of cyclic nucleotides. Since extracellular receptors for many hormones and neurotransmitters utilize cyclic nucleotides as second messengers, the PDEs also regulate cellular responses to these extracellular signals. There are 11 families of PDEs: Ca²⁺/calmodulin-dependent PDEs (PDE1); cGMP-stimulated PDEs (PDE2); cGMP-inhibited PDEs (PDE3); cAMP-specific PDEs (PDE4); cGMP-binding PDEs (PDE5); photoreceptor PDEs (PDE6); high affinity, cAMP-specific PDEs (PDE7); specific PDE (PDE8); high affinity cGMP-specific PDEs (PDE9); and mixed cAMP and cGMP PDEs (PDE10, PDE11).

As stated previously, a reference to any one or more of the herein-described drugs, in particular ibudilast, is meant to encompass, where applicable, any and all enantiomers, mixtures of enantiomers including racemic mixtures, prodrugs, pharmaceutically acceptable salt forms, hydrates (e.g., monohydrates, dihydrates, etc.), different physical forms (e.g., crystalline solids, amorphous solids), metabolites, and the like.

### Formulation Components

In addition to comprising ibudilast, the compositions of the invention may optionally contain one or more additional components as described below.

Excipients/Carriers

In addition to ibudilast, the compositions of the invention for inhibiting glial priming and/or treating pain may further comprise one or more pharmaceutically acceptable excipients or carriers. Exemplary excipients include, without limitation, polyethylene glycol (PEG), hydrogenated castor oil (HCO), cremophors, carbohydrates, starches (e.g., corn starch), inorganic salts, antimicrobial agents, antioxidants, binders/fillers, surfactants, lubricants (e.g., calcium or magnesium stearate), glidants such as talc, disintegrants, diluents, buffers, acids, bases, film coats, combinations thereof, and the like.

A composition of the invention may include one or more carbohydrates such as a sugar, a derivatized sugar such as an alditol, aldonic acid, an esterified sugar, and/or a sugar polymer. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like.

Also suitable for use in the compositions of the invention are potato and corn-based starches such as sodium starch glycolate and directly compressible modified starch.

Further representative excipients include inorganic salt or buffers such as citric acid, sodium chloride, potassium chloride, sodium sulfate, potassium nitrate, sodium phosphate monobasic, sodium phosphate dibasic, and combinations thereof.

A composition comprising ibudilast may also include an antimicrobial agent, e.g., for preventing or deterring microbial growth. Non-limiting examples of antimicrobial agents suitable for the present invention include benzalkonium chloride, benzethonium chloride, benzyl alcohol, cetylpyridinium chloride, chlorobutanol, phenol, phenylethyl alcohol, phenylmercuric nitrate, thimersol, and combinations thereof.

A composition comprising ibudilast may also contain one or more antioxidants. Antioxidants are used to prevent oxidation, thereby preventing the deterioration of the drug(s) or other components of the preparation. Suitable antioxidants for use in the present invention include, for example, ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

Additional excipients include surfactants such as polysorbates, e.g., “Tween 20” and “Tween 80,” and pluronics such as F68 and F88 (both of which are available from BASF, Mount Olive, N.J.), sorbitan esters, lipids (e.g., phospholipids such as lecithin and other phosphatidylcholines, and phosphatidylethanolamines), fatty acids and fatty esters, steroids such as cholesterol, and chelating agents, such as EDTA, zinc and other such suitable cations.

Further, a composition comprising ibudilast may optionally include one or more acids or bases. Non-limiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumarate, and combinations thereof.

The amount of any individual excipient in the composition will vary depending on the role of the excipient, the dosage requirements of the active agent components, and particular needs of the composition. Typically, the optimal amount of any individual excipient is determined through routine experimentation, i.e., by preparing compositions containing varying amounts of the excipient (ranging from low to high), examining the stability and other parameters, and then determining the range at which optimal performance is attained with no significant adverse effects.

Generally, however, the excipient will be present in the composition in an amount of about 1% to about 99% by weight, preferably from about 5% to about 98% by weight, more preferably from about 15 to about 95% by weight of the excipient. In general, the amount of excipient present in a 3,4,6-substituted pyridazine composition is selected from the following: at least about 2%, 5%, 10%, 15%, 20%, 25%, 30%, 35%, 40%, 45%, 50%, 55%, 60%, 65%, 70%, 75%, 80%, 85%, 90%, or even 95% by weight.

These foregoing pharmaceutical excipients along with other excipients are described in “Remington: The Science & Practice of Pharmacy”, 19^{th} ed., Williams & Williams, (1995), the “Physician's Desk Reference”, 52^{nd} ed., Medical Economics, Montvale, N.J. (1998), and Kibbe, A. H., Handbook of Pharmaceutical Excipients, 3^{rd} Edition, American Pharmaceutical Association, Washington, D.C., 2000.

Other Actives

A described above, the formulation (or kit) in accordance with the invention may contain, in addition to ibudilast, one or more other glial attenuators, or other agents effective in treating pain or opioid withdrawal syndrome, including, but not limited to, analgesics, NSAIDs, antiemetics, antidiarrheals, alpha-2-antagonists, benzodiazepines, anticonvulsants, antidepressants, and insomnia therapeutics. Such actives include Minocycline, Fluorocitrate, MW01-5-188WH, Propentofylline (also a PDE inhibitor), Pentoxyfylline (also a PDE inhibitor), Rolipram (also a PDE inhibitor), IL-10, IL-1 receptor antagonist(s), TNF-receptor antagonist(s) including sTNFR, MAP-kinase inhibitor(s), Yohimbine, glial cell chloride antagonists, caspase inhibitors, MMP inhibitors, cannabinoid receptor (e.g., type 2) agonists, arundic acid, statins, thalidomide and related analogs, morphine, oxycodone, and related opiates, buprenorphine, naloxone, methadone, levomethadyl acetate, L-alpha acetylmethadol (LAAM), hydroxyzine, diphenoxylate, atropine, chlordiazepoxide, carbamazepine, mianserin, benzodiazepine, phenoziazine, disulfuram, acamprosate, topiramate, ondansetron, sertraline, bupropion, amantadine, amiloride, isradipine, tiagabine, baclofen, propranolol, tricyclic antidepressants, desipramine, carbamazepine, valproate, lamotrigine, doxepin, fluoxetine, imipramine, moclobemide, nortriptyline, paroxetine, sertraline, tryptophan, venlafaxine, trazodone, quetiapine, zolpidem, zopiclone, zaleplon, gabapentin, memantine, pregabalin, cannabinoids, tramadol, duloxetine, milnacipran, naltrexone, paracetamol, metoclopramide, loperamide, clonidine, lofexidine, and diazepam.

The opiates, morphine and oxycodone, elicit their effects by activating opiate receptors that are widely distributed throughout the brain and body. Once an opiate reaches the brain, it quickly activates the opiate receptors found in many brain regions and produces an effect that correlates with the area of the brain involved. There are several types of opiate receptors, including the delta, mu, and kappa receptors. Opiates and endorphins function to block pain signals by binding to the mu receptor site.

Gabapentin, also known as Neurontin®, is structurally related to the neurotransmitter GABA. Although structurally related to GABA, gabapentin does not interact with GABA receptors, is not converted metabolically into GABA or a GABA agonist, and is not an inhibitor of GABA uptake or degradation. Gabapentin has no activity at GABAA or GABAB receptors of GABA uptake carriers of the brain, but instead interacts with a high-affinity binding site in brain membranes (an auxiliary subunit of voltage-sensitive Ca²⁺ channels). The exact mechanism of action is unknown, only that its physiological site of action is the brain. The structure of gabapentin allows it to pass freely through the blood-brain barrier. In vitro, gabapentin has many pharmacological actions including modulating the action of the GABA synthetic enzyme, increasing non-synaptic GABA responses from neural tissue, and reduction of the release of several mono-amine neurotransmitters. Daily dosages of gabapentin typically range from about 600 to 2400 mg/day, more preferably from about 900 to 1800 mg/day, and are administered in divided doses, for example, three times a day. Conventional unit dosage forms are 300 or 400 mg capsules or 600 or 800 mg tablets.

The active agent, memantine, is a receptor antagonist. Memantine is believed to function as a low to moderate affinity uncompetitive (open-channel) NMDA receptor antagonist which binds to the NMDA receptor-operated cation channels. Recommended daily dosage amounts typically range from about 5 mg to 20 mg.

The cannabinoids, e.g., tetrahydrocannabinol, bind to the cannabinoid receptor referred to as CB₁. CB₁ receptors are found in brain and peripheral tissues; CB₁ receptors are present in high quantities in the central nervous system, exceeding the levels of almost all neurotransmitter receptors. An additional cannabinoid receptor subtype termed ‘CB2’ has also been identified. See, e.g., Martin, B. R., et al., The Journal of Supportive Oncology, Vol. 2, Number 4, July/August 2004.

Although its mechanism of action has not yet been fully elucidated, the opioid, tramadol, is believed to work through modulation of the GABAergic, noradrenergic and serotonergic systems. Tramadol, and its metabolite, known as M1, have been found to bind to 1-opioid receptors (thus exerting its effect on GABAergic transmission), and to inhibit re-uptake of 5-HT and noradrenaline. The second mechanism is believed to contribute since the analgesic effects of tramadol are not fully antagonized by the 1-opioid receptor antagonist naloxone. Typical daily dosages range from about 50 to 100 milligrams every 4 to 6 hours, with a total daily dosage not to exceed 400 milligrams.

Lamotrigine is a phenyltriazine that stabilizes neuronal membranes by blocking voltage-sensitive sodium channels, which inhibit glutamate and aspartate (excitatory amino acid neurotransmitter) release. The daily dosage of lamotrigine typically ranges from 25 milligrams per day to 500 mg per day. Typical daily dosage amounts include 50 mg per day, 100 mg per day, 150 mg per day, 200 mg per day, 300 mg per day, and 500 mgs per day, not exceed 700 mgs per day.

Carbamazepine acts by blocking voltage-sensitive sodium channels. Typical adult dosage amounts range from 100-200 milligrams one or two times daily, to an increased dosage of 800-1200 milligrams daily generally administered in 2-3 divided doses.

Duloxetine is a potent inhibitor of neuronal uptake of serotonin and norephinephrine and a weak inhibitor of dopamine re-uptake. Typical daily dosage amounts range from about 40 to 60 milligrams once daily, or 20 to 30 milligrams twice daily.

Milnacipran acts as a serotonin and norepinephrine reuptake inhibitor. Daily dosage amounts typically range from about 50 to 100 milligrams once or twice daily.

The dosage amounts provided above are meant to be merely guidelines; the precise amount of a secondary active agent to be administered during combination therapy with ibudilast will, of course, be adjusted accordingly and will depend upon factors such as intended patient population, the particular pain symptom or condition to be treated, potential synergies between the active agents administered, and the like, and will readily be determined by one skilled in the art based upon the guidance provided herein.

Sustained Delivery Formulations

The compositions may also be formulated in order to improve stability and extend the half-life of ibudilast. For example, ibudilast may be delivered in a sustained-release formulation. Controlled or sustained-release formulations are prepared by incorporating ibudilast into a carrier or vehicle such as liposomes, nonresorbable impermeable polymers such as ethylenevinyl acetate copolymers and Hytrel® copolymers, swellable polymers such as hydrogels, or resorbable polymers such as collagen and certain polyacids or polyesters such as those used to make resorbable sutures. Additionally, ibudilast can be encapsulated, adsorbed to, or associated with, particulate carriers. Examples of particulate carriers include those derived from polymethyl methacrylate polymers, as well as microparticles derived from poly(lactides) and poly(lactide-co-glycolides), known as PLG. See, e.g., Jeffery et al., Pharm. Res. (1993) 10:362-368; and McGee et al., J. Microencap. (1996).

### Delivery Forms

The compositions comprising ibudilast described herein encompass all types of formulations, and in particular, those that are suited for systemic or intrathecal administration. Oral dosage forms include tablets, lozenges, capsules, syrups, oral suspensions, emulsions, granules, and pellets. Alternative formulations include aerosols, transdermal patches, gels, creams, ointments, suppositories, powders or lyophilates that can be reconstituted, as well as liquids. Examples of suitable diluents for reconstituting solid compositions, e.g., prior to injection, include bacteriostatic water for injection, dextrose 5% in water, phosphate-buffered saline, Ringer's solution, saline, sterile water, deionized water, and combinations thereof. With respect to liquid pharmaceutical compositions, solutions and suspensions are envisioned. Preferably, a composition comprising ibudilast is one suited for oral administration.

In turning now to oral delivery formulations, tablets can be made by compression or molding, optionally with one or more accessory ingredients or additives. Compressed tablets are prepared, for example, by compressing in a suitable tabletting machine, the active ingredients in a free-flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) and/or surface-active or dispersing agent.

Molded tablets are made, for example, by molding in a suitable tabletting machine, a mixture of powdered compounds moistened with an inert liquid diluent. The tablets may optionally be coated or scored, and may be formulated so as to provide slow or controlled release of the active ingredients, using, for example, hydroxypropylmethyl cellulose in varying proportions to provide the desired release profile. Tablets may optionally be provided with a coating, such as a thin film, sugar coating, or an enteric coating to provide release in parts of the gut other than the stomach. Processes, equipment, and toll manufacturers for tablet and capsule making are well-known in the art.

Formulations for topical administration in the mouth include lozenges comprising the active ingredients, generally in a flavored base such as sucrose and acacia or tragacanth and pastilles comprising the active ingredients in an inert base such as gelatin and glycerin or sucrose and acacia.

A pharmaceutical composition for topical administration may also be formulated as an ointment, cream, suspension, lotion, powder, solution, paste, gel, spray, aerosol or oil.

Alternatively, the formulation may be in the form of a patch (e.g., a transdermal patch) or a dressing such as a bandage or adhesive plaster impregnated with active ingredients and optionally one or more excipients or diluents. Topical formulations may additionally include a compound that enhances absorption or penetration of the ingredients through the skin or other affected areas, such as dimethylsulfoxidem bisabolol, oleic acid, isopropyl myristate, and D-limonene, to name a few.

For emulsions, the oily phase is constituted from known ingredients in a known manner. While this phase may comprise merely an emulsifier (otherwise known as an emulgent), it desirably comprises a mixture of at least one emulsifier with a fat and/or an oil. Preferably, a hydrophilic emulsifier is included together with a lipophilic emulsifier that acts as a stabilizer. Together, the emulsifier(s) with or without stabilizer(s) make up the so-called emulsifying wax, and the wax together with the oil and/or fat make up the so-called emulsifying ointment base which forms the oily dispersed phase of cream formulations. Illustrative emulgents and emulsion stabilizers include Tween 60, Span 80, cetostearyl alcohol, myristyl alcohol, glyceryl monostearate and sodium lauryl sulfate.

Formulations for rectal administration are typically in the form of a suppository with a suitable base comprising, for example, cocoa butter or a salicylate.

Formulations suitable for vaginal administration generally take the form of a suppository, tampon, cream, gel, paste, foam or spray.

Formulations suitable for nasal administration, wherein the carrier is a solid, include a coarse powder having a particle size, for example, in the range of about 20 to about 500 microns. Such a formulation is typically administered by rapid inhalation through the nasal passage, e.g., from a container of the powder held in proximity to the nose. Alternatively, a formulation for nasal delivery may be in the form of a liquid, e.g., a nasal spray or nasal drops.

Aerosolizable formulations for inhalation may be in dry powder form (e.g., suitable for administration by a dry powder inhaler), or, alternatively, may be in liquid form, e.g., for use in a nebulizer. Nebulizers for delivering an aerosolized solution include the AERx™ (Aradigm), the Ultravent® (Mallinkrodt), and the Acorn II® (Marquest Medical Products). A composition of the invention may also be delivered using a pressurized, metered dose inhaler (MDI), e.g., the Ventolin® metered dose inhaler, containing a solution or suspension of a combination of drugs as described herein in a pharmaceutically inert liquid propellant, e.g., a chlorofluorocarbon or fluorocarbon.

Formulations suitable for parenteral administration include aqueous and non-aqueous isotonic sterile solutions suitable for injection, as well as aqueous and non-aqueous sterile suspensions.

Parenteral formulations are optionally contained in unit-dose or multi-dose sealed containers, for example, ampoules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets of the types previously described.

A formulation for use with the invention may also be a sustained release formulation, such that each of the drug components is released or absorbed slowly over time, when compared to a non-sustained release formulation. Sustained release formulations may employ pro-drug forms of the active agent, delayed-release drug delivery systems such as liposomes or polymer matrices, hydrogels, or covalent attachment of a polymer such as polyethylene glycol to the active agent.

In addition to the ingredients particularly mentioned above, the formulations may optionally include other agents conventional in the pharmaceutical arts and particular type of formulation being employed, for example, for oral administration forms, the composition for oral administration may also include additional agents as sweeteners, thickeners or flavoring agents.

The compositions comprising ibudilast may also be prepared in a form suitable for veterinary applications.

### Kits

Also provided herein is a kit containing a composition comprising ibudilast accompanied by instructions for use, e.g., in treating pain. In addition, the kit may optionally contain one or more other glial attenuators or other agents for treating pain or opioid withdrawal syndrome.

For example, in instances in which each of the drugs themselves are administered as individual or separate dosage forms, the kit comprises ibudilast in addition to each of the drugs making up the composition of the invention, along with instructions for use. Ibudilast and one or more opioids or other agents may be present in the same or separate compositions. The drug components may be packaged in any manner suitable for administration, so long as the packaging, when considered along with the instructions for administration, clearly indicates the manner in which each of the drug components is to be administered.

For example, for an illustrative kit comprising ibudilast and morphine, the kit may be organized by any appropriate time period, such as by day. As an example, for Day 1, a representative kit may comprise unit dosages of each of ibudilast and morphine. If each of the drugs is to be administered twice daily, then the kit may contain, corresponding to Day 1, two rows of unit dosage forms of each of ibudilast and morphine, along with instructions for the timing of administration. Alternatively, if one or more of the drugs differs in the timing or quantity of unit dosage form to be administered in comparison to the other drug members of the combination, then such would be reflected in the packaging and instructions. Various embodiments according to the above may be readily envisioned, and would of course depend upon the particular combination of drugs, in addition to ibudilast, employed for treatment, their corresponding dosage forms, recommended dosages, intended patient population, and the like. The packaging may be in any form commonly employed for the packaging of pharmaceuticals, and may utilize any of a number of features such as different colors, wrapping, tamper-resistant packaging, blister packs, desiccants, and the like.

### Method of Administration

As set forth above, the present invention encompasses a method of preventing or diminishing pain amplification in a mammalian subject following an initial glial activating event by administering a therapeutically effective dosage of ibudilast. Such administering is effective to decrease the amount of pain experienced by the subject, i.e., to result in significant attenuation or elimination of pain, during a subsequent pain-evoking event.

In certain embodiments, the subject is administered an effective amount of ibudilast at about the same time as or shortly before or after the initial glial-activating event. Preferably the subject is administered a therapeutically effective amount of ibudilast about 1-5 days before and/or about 1-5 days after the initial glial-activating event.

Therapeutic amounts can be empirically determined and will vary with the particular condition being treated, the subject, and the particular efficacy and toxicity of each of the active agents contained in the composition. The actual dose to be administered will vary depending upon the age, weight, and general condition of the subject as well as the severity of the condition being treated, the judgment of the health care professional, and particular mode of administration.

The method of the invention may, in certain instances, comprise a step of selecting a subject experiencing pain prior to administering thereto ibudilast. Such subjects are typically selected from those suffering from physical trauma associated with inflammation, injury, surgery, or disease.

The method of the invention may be effective to not only significantly attenuate pain during an initial pain-evoking event, but may also diminish or prevent amplification of pain during a subsequent pain episode, and prevent the development of chronic pain.

Ibudilast may also be administered in combination with one or more other glial attenuators or additional agents effective for treating pain or opioid withdrawal syndrome. Exemplary agents include other analgesics, non-steroidal anti-inflammatory drugs (NSAIDs), antiemetics, antidiarrheals, alpha-2-antagonists, benzodiazepines, anticonvulsants, antidepressants, and insomnia therapeutics.

Preferred methods of delivery of therapeutic formulations comprising ibudilast for the treatment of pain include systemic and localized delivery, i.e., directly into the central nervous system. Such routes of administration include but are not limited to, oral, intra-arterial, intrathecal, intraspinal, intramuscular, subcutaneous, intraperitoneal, intravenous, intranasal, and inhalation routes.

More particularly, a formulation containing ibudilast of the present invention may be administered for therapy by any suitable route, including without limitation, oral, rectal, nasal, topical (including transdermal, aerosol, buccal and sublingual), vaginal, parenteral (including subcutaneous, intramuscular, intravenous and intradermal), intrathecal, and pulmonary. The preferred route will, of course, vary with the condition and age of the recipient, the particular neuralgia-associated syndrome being treated, and the specific combination of drugs employed.

One preferred mode of administration for delivery of ibudilast is directly to neural tissue such as peripheral nerves, the retina, dorsal root ganglia, neuromuscular junction, as well as the CNS, e.g., to target spinal cord glial cells by injection into, e.g., the ventricular region, as well as to the striatum (e.g., the caudate nucleus or putamen of the striatum), spinal cord and neuromuscular junction, with a needle, catheter or related device, using neurosurgical techniques known in the art, such as by stereotactic injection (see, e.g., Stein et al., J. Virol. 73:3424-3429, 1999; Davidson et al., PNAS 97:3428-3432, 2000; Davidson et al., Nat. Genet. 3:219-223, 1993; and Alisky and Davidson, Hum. Gene Ther. 11:2315-2329, 2000).

A particularly preferred method for targeting spinal cord glia is by intrathecal delivery, rather than into the cord tissue itself.

Another preferred method for administering the compositions comprising ibudilast of the invention is by delivery to dorsal root ganglia (DRG) neurons, e.g., by injection into the epidural space with subsequent diffusion to DRG. For example, an ibudilast-based composition can be delivered via intrathecal cannulation under conditions where ibudilast is diffused to DRG. See, e.g., Chiang et al., Acta Anaesthesiol. Sin. (2000) 38:31-36; Jain, K. K., Expert Opin. Investig. Drugs (2000) 9:2403-2410.

Yet another mode of administration to the CNS uses a convection-enhanced delivery (CED) system. In this way, ibudilast can be delivered to many cells over large areas of the CNS. Any convection-enhanced delivery device may be appropriate for delivery of ibudilast. In a preferred embodiment, the device is an osmotic pump or an infusion pump. Both osmotic and infusion pumps are commercially available from a variety of suppliers, for example Alzet Corporation, Hamilton Corporation, Alza, Inc., Palo Alto, Calif.). Typically, a composition comprising ibudilast of the invention is delivered via CED devices as follows. A catheter, cannula or other injection device is inserted into CNS tissue in the chosen subject. Stereotactic maps and positioning devices are available, for example from ASI Instruments, Warren, Mich. Positioning may also be conducted by using anatomical maps obtained by CT and/or MRI imaging to help guide the injection device to the chosen target. For a detailed description regarding CED delivery, see U.S. Pat. No. 6,309,634, incorporated herein by reference in its entirety.

A composition comprising ibudilast, when comprising more than one active agent, may be administered as a single combination composition comprising a combination of ibudilast and at least one additional active agent effective in the treatment of pain. In terms of patient compliance and ease of administration, such an approach is preferred, since patients are often adverse to taking multiple pills or dosage forms, often multiple times daily, over the duration of treatment. Alternatively, albeit less preferably, the combination of the invention is administered as separate dosage forms. In instances in which the drugs comprising the therapeutic composition of the invention are administered as separate dosage forms and co-administration is required, ibudilast and each of the additional active agents may be administered simultaneously, sequentially in any order, or separately.

### Dosages

Therapeutic amounts can be empirically determined by those skilled in the art and will vary with the particular condition being treated, the subject, and the efficacy and toxicity of each of the active agents contained in the composition. The actual dose to be administered will vary depending upon the age, weight, and general condition of the subject as well as the severity of the condition being treated, the judgment of the health care professional, and the particular combination of ibudilast, and any other agents being administered.

Therapeutically effective amounts can be determined by those skilled in the art, and will be adjusted to the requirements of each particular case. Generally, a therapeutically effective amount of ibudilast will range from a total daily dosage, for example in humans, of about 0.1 and 500 mg/day, more preferably, in an amount between 1 and 200 mg/day, 1 and 100 mg/day, 1 and 40 mg/day, or 1 and 20 mg/day, administered as either a single dosage or as multiple dosages.

Depending upon the dosage amount and precise condition to be treated, administration can be one, two, or three times daily, or even more, for a time course of one day to several days, weeks, months, and even years, and may even be for the life of the patient. Intermittent dosing may also be employed, e.g., in response to pain, with a maximal dose not to be exceeded as recommended by the practicing physician. Illustrative dosing regimes will last a period of at least about a week, from about 1-4 weeks, from 1-3 months, from 1-6 months, from 1-50 weeks, from 1-12 months, or longer.

### Pain Models

The ability of ibudilast to diminish amplification of pain following a glial-activating event can be evaluated by any of the standard pain models known in the art. Examples of such models are as follows.

Tail Flick Model: The tail-flick test (D'Amour et al. (1941) J. Pharmacol. Exp. and Ther. 72:74-79) is a model of acute pain. A towel-wrapped rat is placed on a test stage such that a focused light source beams on the dorsal surface of the rat's tail. A photosensor is present on the test stage located opposite the light source and below the rat's tail. To begin the test, the rat's tail blocks the light, thus preventing the light reaching the photosensor. Latency measurement begins with the activation of the light source. When a rat moves or flicks its tail, the photosensor detects the light source and stops the measurement. The test measures the period of time (duration) that the rat's tail remains immobile (latent). Rats are tested prior to administration thereto of a compound of interest and then at various times after such administration. The light source is set to an intensity that produced a tail response latency of about 3 seconds when applied to the tails of rats to which no compound has been administered.

Rat Tail Immersion Model: The rat tail immersion assay is also a model of acute pain. A rat is loosely held in hand while covered with a small folded thin cotton towel with its tail exposed. The tip of the tail is dipped into a, e.g., 52° C. water bath to a depth of two inches. The rat responds by either wiggling of the tail or withdrawal of the tail from the water; either response is scored as the behavioral end-point. Rats are tested for a tail response latency (TRL) score prior to administration thereto of a compound of interest and then retested for TRL at various times after such administration.

Carrageenan-induced Paw Hyperalgesia Model: The carrageenan paw hyperalgesia test is a model of inflammatory pain. A subcutaneous injection of carrageenan is made into the left hindpaws of rats. The rats are treated with a selected agent before, e.g., 30 minutes, the carrageenan injection or after, e.g., two hours after, the carrageenan injection. Paw pressure sensitivity for each animal is tested with an analgesymeter three hours after the carrageenan injection. See, Randall et al. (1957) Arch. Int. Pharmacodyn. 111:409-419.

The effects of selected agents on carrageenan-induced paw edema can also be examined. This test (see, Vinegar et al. (1969) J. Phamacol. Exp. Ther. 166:96-103) allows an assessment of the ability of a compound to reverse or prevent the formation of edema evoked by paw carrageenan injection. The paw edema test is carried out using a plethysmometer for paw measurements. After administration of a selected agent, a carrageenan solution is injected subcutaneously into the lateral foot pad on the plantar surface of the left hind paw. At three hours post-carrageenan treatment, the volume of the treated paw (left) and the un-treated paw (right) is measured using a plethysmometer.

Formalin Behavioral Response Model: The formalin test is a model of acute, persistent pain. Response to formalin treatment is biphasic (Dubuisson et al. (1977) Pain 4:161-174). The Phase I response is indicative of a pure nociceptive response to the irritant. Phase 2, typically beginning 20 to 60 minutes following injection of formalin, is thought to reflect increased sensitization of the spinal cord.

Von Frey Filament Test: The effect of compounds on mechanical allodynia can be determined by the von Frey filament test in rats with a tight ligation of the L-5 spinal nerve: a model of painful peripheral neuropathy. The surgical procedure is performed as described by Kim et al. (1992) Pain 50:355-363. A calibrated series of von Frey filaments are used to assess mechanical allodynia (Chaplan et al. (1994) J. Neurosci. Methods 53:55-63). Filaments of increasing stiffness are applied perpendicular to the midplantar surface in the sciatic nerve distribution of the left hindpaw. The filaments are slowly depressed until bending occurred and are then held for 4-6 seconds. The filament application order and number of trials were determined by the up-down method of Dixon (Chaplan et al., supra). Flinching and licking of the paw and paw withdrawal on the ligated side are considered positive responses.

Chronic Constriction Injury: Heat and cold allodynia responses can be evaluated as described below in rats having a chronic constriction injury (CCI). A unilateral mononeuropathy is produced in rats using the chronic constriction injury model described in Bennett et al. (1988) Pain 33:87-107.

CCI is produced in anesthetized rats as follows. The lateral aspect of each rat's hind limb is shaved and scrubbed with Nolvasan. Using aseptic techniques, an incision is made on the lateral aspect of the hind limb at the mid-thigh level. The biceps femoris is bluntly dissected to expose the sciatic nerve. On the right hind limb of each rat, four loosely tied ligatures (for example, Chromic gut 4.0; Ethicon, Johnson and Johnson, Somerville, N.J.) are made around the sciatic nerve approximately 1-2 mm apart. On the left side of each rat, an identical dissection is performed except that the sciatic nerve is not ligated (sham). The muscle is closed with a continuous suture pattern with, e.g., 4-0 Vicryl (Johnson and Johnson, Somerville, N.J.) and the overlying skin is closed with wound clips. The rats are ear-tagged for identification purposes and returned to animal housing.

Radiant Heat Model: CCI rats are tested for thermal hyperalgesia at least 10 days post-op. The test apparatus consists of an elevated heated (80-82° F.) glass platform. Eight rats at a time, representing all testing groups, are confined individually in inverted plastic cages on the glass floor of the platform at least 15 minutes before testing. A radiant heat source placed underneath the glass is aimed at the plantar hind paw of each rat. The application of heat is continued until the paw is withdrawn (withdrawal latency) or the time elapsed is 20 seconds. This trial is also applied to the sham operated leg. Two to four trials are conducted on each paw, alternately, with at least 5 minutes interval between trials. The average of these values represents the withdrawal latency.

Cold Allodynia Model: The test apparatus and methods of behavioral testing is described in Gogas et al. (1997) Analgesia 3:111-118. The apparatus for testing cold allodynia in neuropathic (CCI) rats consists of a Plexiglass chamber with a metal plate 6 cm from the bottom of the chamber. The chamber is filled with ice and water to a depth of 2.5 cm above the metal plate, with the temperature of the bath maintained at 0-4° C. throughout the test.

Chung Model of Rat Neuropathic Pain: Heat and cold allodynia responses as well as mechanical allodynia sensations can be evaluated as described below in rats following spinal nerve injury (e.g. ligation, transaction). Details are as initially described in S H Kim and J M Chung, Pain (1992) 50:355-363.

The Hargreaves Test: The Hargreaves test (Hargreaves et al., Pain (1998) 32:77-88) is also a radiant heat model for pain. CCI rats are tested for thermal hyperalgesia at least 10 days post-op. The test apparatus consists of an elevated heated (80-82° F.) glass platform. Eight rats at a time, representing all testing groups, are confined individually in inverted plastic cages on the glass floor of the platform at least 15 minutes before testing. A radiant heat source placed underneath the glass is aimed at the plantar hind paw of each rat. The application of heat is continued until the paw is withdrawn (withdrawal latency) or the time elapsed is 20 seconds. This trial is also applied to the sham operated leg. Two to four trials are conducted on each paw, alternately, with at least 5 minutes interval between trials. The average of these values represents the withdrawal latency.

### III. EXPERIMENTAL

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### Example 1

### Treatment with Ibudilast to Prevent Pain Amplification from Glial Priming

Rats were treated with 50 mg/kg ibudilast orally (p.o.) by gavage 2 days prior and 5 days after laparotomy surgery (injury inducing spinal cord glial activation). Two weeks after surgery, all rats received 100 mg/kg cyclophosphamide (CP) intraperitoneally (I.p.). In the kidneys, CP turns into an acrolein derivative (mustard gas) that causes sterile cystitis and referred pain in the hindpaws of rats. The von Frey test was used to compare the responses to pain of untreated rats and rats treated with ibudilast. Baseline von Frey values were obtained one day before rats were injected with CP. Later timepoints are relative to the CP injection reference time point.

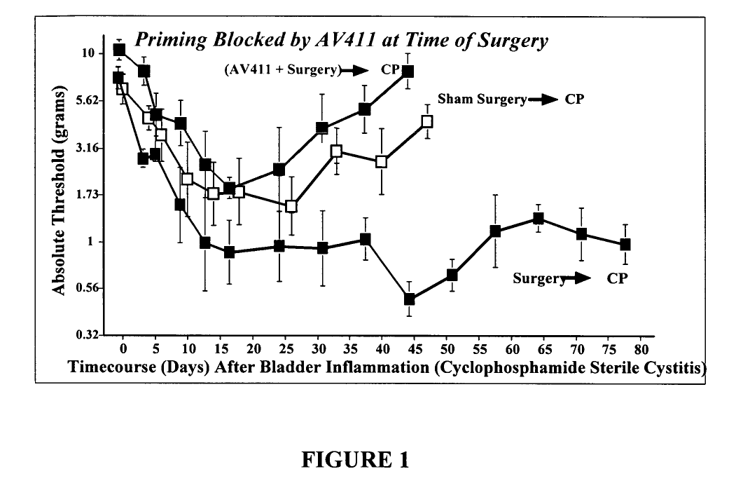

Rats subjected to a prior laparotomy and then subsequent CP injection showed greatly enhanced pain for over 47 days (experiment stopped at this time) compared to control rats that had not had the laparotomy. Pain in untreated control animals that were anesthetized 2 weeks prior to the CP injection, but had not had surgery, resolved in about 3 weeks, whereas cystitis pain in untreated rats that previously received a laparotomy continued for more than 80 days. In contrast, rats that received ibudilast at the time of the laparotomy behaved no differently than rats that received Vehicle instead of cyclcophosphamide (see FIG. 1). Therefore, a pre-emptive treatment with ibudilast at about the time of surgery prevented pain amplification from a later challenge.

Thus, the use of ibudilast to prevent amplified pain responses resulting from glial priming is described. Ibudilast can be used to decrease pain from a new challenge after an initial glial activating event or to prevent the development of chronic pain. Although preferred embodiments of the subject invention have been described in some detail, it is understood that obvious variations can be made without departing from the spirit and the scope of the invention as claimed herein.

All references cited herein, including patents, patent applications and other publications, are hereby incorporated by reference in their entireties.

What is claimed is:

1. A method for inhibiting glial priming in a subject that has experienced a glial-activating event, comprising administering to the subject an effective amount of ibudilast.

2. The method of claim 1, wherein the subject is human.

3. The method of claim 1, wherein the ibudilast is administered systemically.

4. The method of claim 1, wherein the ibudilast is administered centrally.

5. The method of claim 4, wherein the ibudilast is administered intrathecally.

6. The method of claim 1, wherein multiple therapeutically effective doses of the ibudilast are administered to the subject.

7. The method of claim 1, wherein the glial-activating event is tissue injury, infection or inflammation.

8. The method of claim 1, wherein the ibudilast is administered at about the same time as the glial-activating event.

9. The method of claim 1, wherein the ibudilast is administered before the glial-activating event.

10. The method of claim 1, wherein the ibudilast is administered after the glial-activating event.

11. The method of claim 1, wherein the ibudilast is administered before and after the glial-activating event.

12. The method of claim 1, wherein the ibudilast is administered at about the same time as the glial-activating event and after the glial-activating event.

13. The method of claim 1, wherein the ibudilast is administered about 1-5 days before and about 1-5 days after the glial-activating event.

14. A method for preventing or diminishing amplified pain resulting from glial priming after a first glial-activating event, the method comprising administering to a subject in need thereof a therapeutically effective amount of ibudilast, wherein amplified pain after a second glial-activating event is diminished or eliminated.

15. The method of claim 14, wherein the subject is human.

16. The method of claim 14, wherein the ibudilast is administered systemically.

17. The method of claim 14, wherein the ibudilast is administered centrally.

18. The method of claim 17, wherein the ibudilast is administered intrathecally.

19. The method of claim 14, wherein multiple therapeutically effective doses of the ibudilast are administered to the subject.

20. The method of claim 14, wherein the first glial-activating event is tissue injury, infection or inflammation.

21. The method of claim 14, wherein the subject has post-operative pain, injury-related pain, or disease-related pain.

22. The method of claim 14, wherein the subject has chronic pain.

23. The method of claim 14, wherein the subject has neuropathic pain.

24. The method of claim 14, wherein the ibudilast is administered at about the same time as the first glial-activating event.

25. The method of claim 14, wherein the ibudilast is administered before the first glial-activating event.

26. The method of claim 14, wherein the ibudilast is administered after the first glial-activating event.

27. The method of claim 14, wherein the ibudilast is administered before and after the first glial-activating event.

28. The method of claim 14, wherein the ibudilast is administered at about the same time as the first glial-activating event and after the first glial-activating event.

29. The method of claim 14, wherein the ibudilast is administered about 1-5 days before and about 1-5 days after the first glial-activating event.

30. The method of claim 14, further comprising administering ibudilast at about the same time as or after the second glial activating event.

31. The method of claim 14, further comprising administering one or more other glial attenuators.

32. The method of claim 31, wherein the glial attenuator is minocycline.

33. The method of claim 14, further comprising administering one or more other agents selected from the group consisting of analgesics, non-steroidal anti-inflammatory drugs (NSAIDs), antiemetics, antidiarrheals, alpha-2-antagonists, benzodiazepines, anticonvulsants, antidepressants, and insomnia therapeutics.
